(19) United States
(12) Patent Application Publication
(10) Pub. No.: US 2021/0015374 A1
(43) Pub. Date: Jan. 21, 2021

(54) ELECTRONIC BLOOD PRESSURE METER AND HEART FAILURE DETECTOR

(71) Applicant: OMRON HEALTHCARE CO., LTD., Muko-shi (JP)

(72) Inventor: Yukiya SAWANOI, Kyoto (JP)

(73) Assignee: OMRON HEALTHCARE CO., LTD., Muko-shi (JP)

(21) Appl. No.: 17/062,666

(22) Filed: Oct. 5, 2020

Related U.S. Application Data
(63) Continuation of application No. PCT/JP2019/007725, filed on Feb. 28, 2019.

(30) Foreign Application Priority Data
Apr. 20, 2018 (JP) 2018-081746

(51) Int. Cl. A61B 5/0225; A61B 5/022; A61B 5/00; A61B 5/0205

(52) U.S. Cl. CPC A61B 5/0225; A61B 5/02225; A61B 5/746; A61B 5/08; A61B 5/0205; A61B 5/7425; A61B 5/7278

(57) ABSTRACT

An electronic blood pressure meter according to the present invention includes: a cuff pressure control unit capable of changing a pressure of a cuff worn on a measurement site; a pressure detection unit that detects a cuff pressure signal representing the pressure of the cuff; a variation amount calculation unit that obtains a blood pressure variation amount synchronized with respiration based on the cuff pressure signal; a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and an index output unit that refers to the correspondence relation stored in the correspondence relation storage unit and outputs the heart failure index corresponding to the blood pressure variation amount obtained by the variation amount calculation unit.

### CROSS-REFERENCE TO RELATED APPLICATION

This is a continuation application of International Application No. PCT/JP2019/007725, with an International filing date of Feb. 28, 2019, which claims priority of Japanese Patent Application No. 2018-081746 filed on Apr. 20, 2018, the entire content of which is hereby incorporated by reference.

### TECHNICAL FIELD

The present invention relates to an electronic blood pressure meter, and more particularly, to an electronic blood pressure meter that measures a blood pressure at a measurement site using an oscillometric method. The present invention also relates to a heart failure detector that outputs an index representing a relative severity of heart failure.

### BACKGROUND ART

In recent years, there is an increasing need to monitor the condition of heart failure at home. That is, generally speaking, heart failure is acutely exacerbated over time, and a patient suffering from the exacerbated heart failure requires hospitalization. Even if the patient receives hospital treatment, he/she will not recover as he/she used to be before hospitalization, and would gradually deteriorate with repeating in and out of hospital. Heart failure means an organ failure condition, and is hard to cure as described above. However, if the patient receives optimum care before the abovementioned acute exacerbation, the degree of progression of the subsequent deterioration of the condition can be moderated. Therefore, as described above, there is an increasing need to monitor the condition of heart failure at home.

As a device for monitoring the condition of heart failure at home, OptiVol (registered trademark) (manufactured by Medtronic) for measuring impedance in the thorax is known, for example, as described in Non-Patent Literature 1 (Ishimaru et al., “A case report; OptiVol™ is useful modality in early detection and treatment for heart failure”, Heart, 45(3), PP. 321-326, 2013). This device is configured to detect, based on an impedance change, the state of congestion in lung due to the deterioration in the pumping function of the heart.

### SUMMARY OF THE INVENTION

Meanwhile, when the above OptiVol (registered trademark) is used, it is necessary that a doctor implants the device in the body of a patient. For this reason, it cannot be readily used by ordinary people other than doctors.

On the other hand, an electronic blood pressure meter that measures the blood pressure at a measurement site with, for example, an oscillometric method is non-invasive to a subject, so that it can be readily used by ordinary people other than doctors.

In view of this, an object of the present invention is to provide an electronic blood pressure meter that measures the blood pressure at a measurement site using an oscillometric method and can output an index (hereinafter referred to as a “heart failure index” as appropriate) representing the relative severity of heart failure non-invasively to a subject. Another object of the present invention is to provide a heart failure detector that can output such a heart failure index non-invasively to a subject.

In order to achieve the above object, an electronic blood pressure meter according to the present disclosure is an electronic blood pressure meter for measuring a blood pressure at a measurement site using an oscillometric method, the electronic blood pressure meter comprising:

a cuff pressure control unit capable of changing a pressure of a cuff worn on the measurement site;

a pressure detection unit that detects a cuff pressure signal representing the pressure of the cuff;

a variation amount calculation unit that obtains a blood pressure variation amount synchronized with respiration based on the cuff pressure signal;

a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure;

and an index output unit that refers to the correspondence relation stored in the correspondence relation storage unit and outputs the heart failure index corresponding to the blood pressure variation amount obtained by the variation amount calculation unit.

In another aspect, a heart failure detector of the present disclosure is a heart failure detector for outputting an index relatively representing severity of heart failure, the heart failure detector comprising:

a cuff pressure control unit capable of changing a pressure of a cuff worn on a measurement site;

a pressure detection unit that detects a cuff pressure signal representing the pressure of the cuff;

a variation amount calculation unit that obtains a blood pressure variation amount synchronized with respiration based on the cuff pressure signal;

a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and

an index output unit that refers to the correspondence relation stored in the correspondence relation storage unit and outputs the heart failure index corresponding to the blood pressure variation amount obtained by the variation amount calculation unit.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a diagram showing an external appearance of an electronic blood pressure meter according to an embodiment of the present invention when a cuff is wrapped around an upper arm as a measurement site.

FIG. 2 is a block diagram showing a configuration of a control system of the electronic blood pressure meter.

FIG. 3 is a diagram illustrating a predetermined correspondence relation between a blood pressure variation amount and a heart failure index stored in a memory of the electronic blood pressure meter.

FIG. 4 is a diagram showing a schematic operation flow of the electronic blood pressure meter.

FIG. 5 is a diagram illustrating a set of elements included in a central processing unit (CPU) of the electronic blood pressure meter for calculating a blood pressure value and a blood pressure variation amount.

FIG. 6 is a diagram showing a process of calculating a blood pressure value and a blood pressure variation amount using the elements in FIG. 5.

FIG. 7A is a diagram illustrating a cuff pressure signal detected via a pressure sensor of the electronic blood pressure meter. FIG. 7B is a diagram illustrating a signal (HPF output) extracted from the cuff pressure signal through a high-pass filter.

FIG. 8 is a diagram illustrating the signal in FIG. 7B during a pressure decrease process being in an enlarged manner and represented as a pulse wave signal indicating a pulse wave at the measurement site.

FIG. 9 is a diagram showing a train of amplitudes indicated by the pulse wave signal and a first envelope created for the train of amplitudes.

FIG. 10 shows an example in which, in a case where the respiratory period of a subject is known, a train of amplitudes is acquired for each phase of the respiratory period from the pulse wave signal, and envelopes are created for each of the trains of amplitudes for each phase.

FIG. 11 is a diagram showing a method for calculating systolic blood pressure and diastolic blood pressure using a local-maximum envelope and a local-minimum envelope.

FIG. 12 is a diagram showing an example of a blood pressure variation amount determined using the local-maximum envelope and the local-minimum envelope.

FIG. 13 is a diagram showing another example of a blood pressure variation amount determined using the local-maximum envelope and the local-minimum envelope.

FIG. 14 is a diagram showing a content displayed on a display of the electronic blood pressure meter.

FIG. 15 shows a local-maximum envelope EVmax and a local-minimum envelope EVmin created for a patient B by the electronic blood pressure meter on date of admission.

FIG. 16 shows a local-maximum envelope EVmax and a local-minimum envelope EVmin created for the patient B by the electronic blood pressure meter on date of discharge.

FIG. 17 is a scatter diagram showing the relationship between a blood pressure variation amount ΔBP obtained by the electronic blood pressure meter and NT-proBNP (N-terminal pro B-type natriuretic peptide) measured for patients A and B on date of admission and on date of discharge.

FIG. 18A is a scatter diagram showing the relationship between a heart failure index HFI obtained by the electronic blood pressure meter and NT-proBNP which are measured on date of admission and on date of discharge for the patient A.

FIG. 18B is a scatter diagram showing the relationship between a heart failure index HFI obtained by the electronic blood pressure meter and NT-proBNP which are measured on date of admission and on date of discharge for the patient B.

### DESCRIPTION OF EMBODIMENT

An embodiment of the present invention will now be described in detail with reference to the drawings.

### (Schematic Configuration of Electronic Blood Pressure Meter)

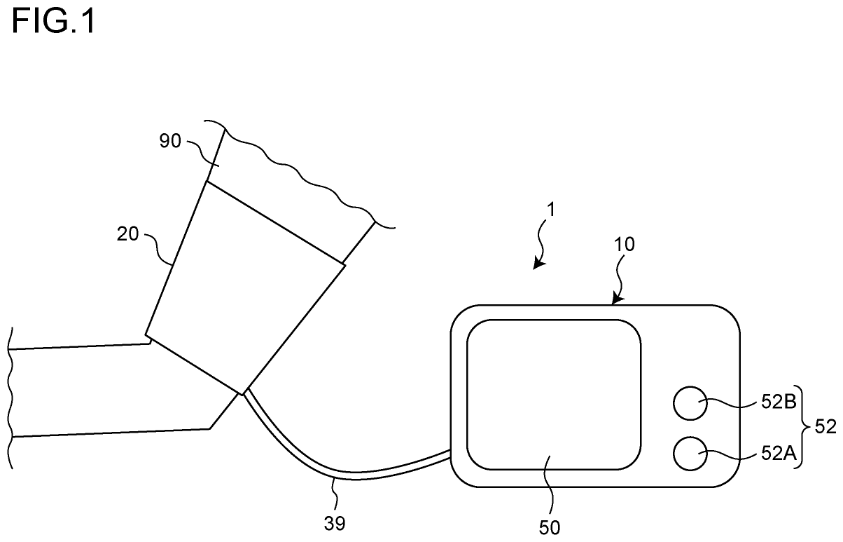

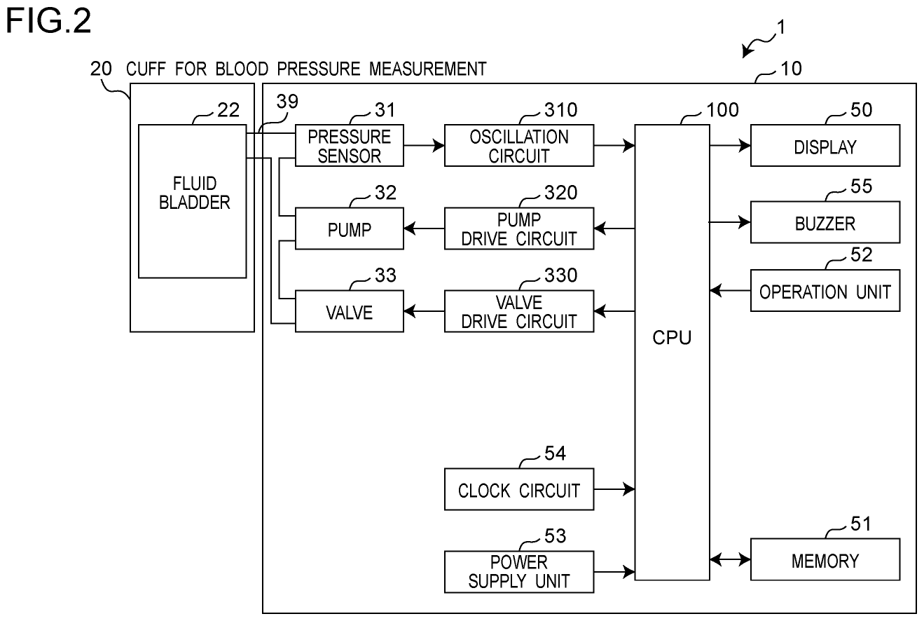

FIG. 1 shows an external appearance of an electronic blood pressure meter (the entire of the electronic blood pressure meter is indicated by reference numeral 1, and the electronic blood pressure meter is hereinafter simply referred to as a “blood pressure meter”) according to an embodiment of the present invention. The blood pressure meter 1 is for home use, and includes a cuff 20 for blood pressure measurement that is to be wrapped around an upper arm 90 as a measurement site of a subject, a main body 10, and a flexible cuff air tube 39 that connects the cuff 20 and the main body 10. The cuff 20 contains a fluid bladder 22 (see FIG. 2) for pressing the upper arm. A display 50 and an operation unit 52 are provided on the front surface of the main body 10.

The display 50 is composed of a liquid crystal display element (LCD) in this example, and electronically displays information regarding blood pressure measurement in accordance with a control signal from a central processing unit (CPU) 100 (see FIG. 2) described later.

The operation unit 52 has a power switch 52A that receives an input of an instruction to turn on or off the power of the blood pressure meter 1, and a start/stop switch 52B that receives an instruction to start or stop the measurement of blood pressure. These switches 52A and 52B input an operation signal according to an instruction from a user to the CPU 100.

As shown in FIG. 2, the main body 10 of the blood pressure meter 1 is equipped with, in addition to the CPU 100, the display 50, and the operation unit 52 described above, a memory 51 serving as a storage unit, a clock circuit 54, a buzzer 55, a power supply unit 53, a pump 32, a valve (electromagnetic control valve) 33, and a pressure sensor 31. Further, the main body 10 is equipped with an oscillation circuit 310 that converts an output from the pressure sensor 31 into a frequency, a pump drive circuit 320 that drives the pump 32, and a valve drive circuit 330 that drives the valve 33. The pump 32, the valve 33, and the pressure sensor 31 are connected to the cuff 20 (containing the fluid bladder 22) via the cuff air tube 39.

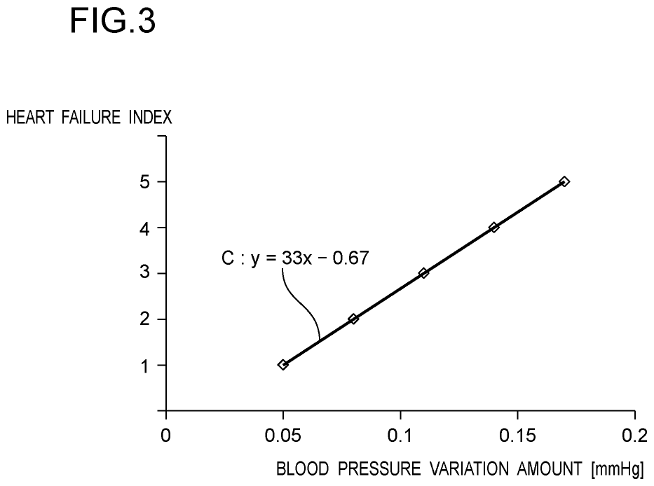

The memory 51 stores data of a program for controlling the blood pressure meter 1, data used for controlling the blood pressure meter 1, setting data for setting various functions of the blood pressure meter 1, and data of measurement results of blood pressure values. Further, the memory 51 is used as a working memory or the like when the program is executed. In this example, the memory 51 stores, as a correspondence relation storage unit, a predetermined correspondence relation C between a blood pressure variation amount and a heart failure index as shown in FIG. 3 (this correspondence relation will be described later).

The CPU 100 shown in FIG. 2 controls driving of the pump 32 and the valve 33 according to an operation signal from the operation unit 52 in accordance with a program for controlling the blood pressure meter 1 stored in the memory 51. Further, the CPU 100 calculates a blood pressure value based on the signal from the pressure sensor 31, and controls the display 50 and the memory 51.

The clock circuit 54 oscillates a clock frequency for the operation of the CPU 100 and counts the current date and time.

The buzzer 55 generates an alarm sound according to the control signal from the CPU 100.

The power supply unit 53 supplies electric power to each unit in the main body 10.

The pump 32 supplies air to the fluid bladder 22 contained in the cuff 20 in order to increase the pressure (cuff pressure) in the fluid bladder 22. The valve 33 is opened and closed to discharge or seal air from or in the fluid bladder 22 to thereby control the cuff pressure. The pump drive circuit 320 drives the pump 32 based on the control signal supplied from the CPU 100. The valve drive circuit 330 opens and closes the valve 33 based on the control signal supplied from the CPU 100.

The pressure sensor 31 and the oscillation circuit 310 operate to detect the cuff pressure. The pressure sensor 31 is, for example, a piezoresistive pressure sensor, and is connected to the pump 32, the valve 33, and the fluid bladder 22 contained in the cuff 20 via the cuff air tube 39. In this example, the oscillation circuit 310 oscillates based on an electric signal value which is based on a change in electric resistance due to the piezoresistive effect from the pressure sensor 31, and outputs a frequency signal having a frequency corresponding to the electric signal value of the pressure sensor 31 to the CPU 100. The CPU 100 obtains a cuff pressure signal representing the cuff pressure based on the frequency signal.

### (Schematic Measurement Operation)

When a blood pressure is measured according to a common oscillometric method, the following operation is generally performed. That is, the cuff is wrapped around the measurement site (for example, arm) of the subject in advance, and during measurement, the pump and valve are controlled so as to increase the cuff pressure to be higher than the maximum blood pressure, and gradually reduce the cuff pressure thereafter. During the pressure decrease process, the cuff pressure is detected by the pressure sensor, and variations in the arterial volume that occur in the artery at the measurement site are extracted as a pulse wave signal. Based on changes (mainly rising edges and falling edges) in the amplitude of the pulse wave signal, which corresponds to changes in the cuff pressure at that time, maximum blood pressure (systolic blood pressure) and minimal blood pressure (diastolic blood pressure) are calculated.

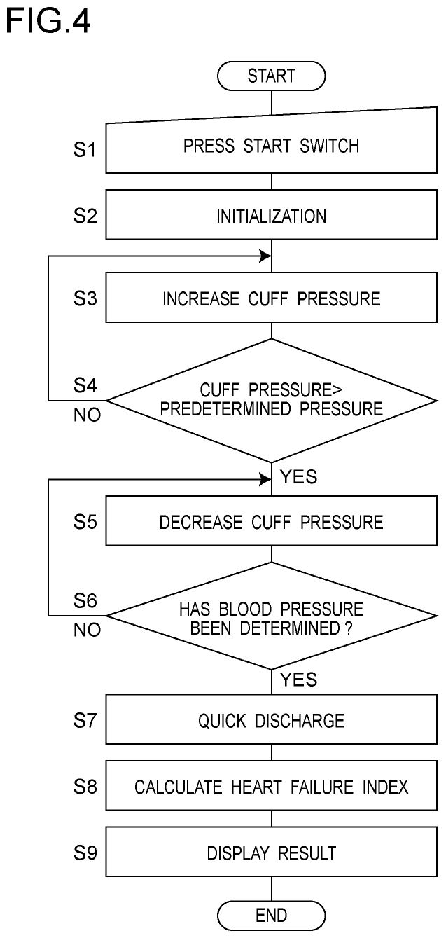

In the blood pressure meter 1, the blood pressure value of the subject and a heart failure index relatively representing the severity of heart failure are measured by the CPU 100 using an oscillometric method according to the flow of FIG. 4.

Specifically, as shown in step S1 of FIG. 4, when the start/stop switch 52B is pressed while the power switch 52A is on, the blood pressure meter 1 starts blood pressure measurement. When blood pressure measurement is started, the CPU 100 initializes the memory region for processing, and outputs a control signal to the valve drive circuit 330, in step S2. The valve drive circuit 330 opens the valve 33 so as to discharge air in the fluid bladder 22 of the cuff 20 based on the control signal. Then, control for adjusting the pressure sensor 31 to 0 mmHg is performed.

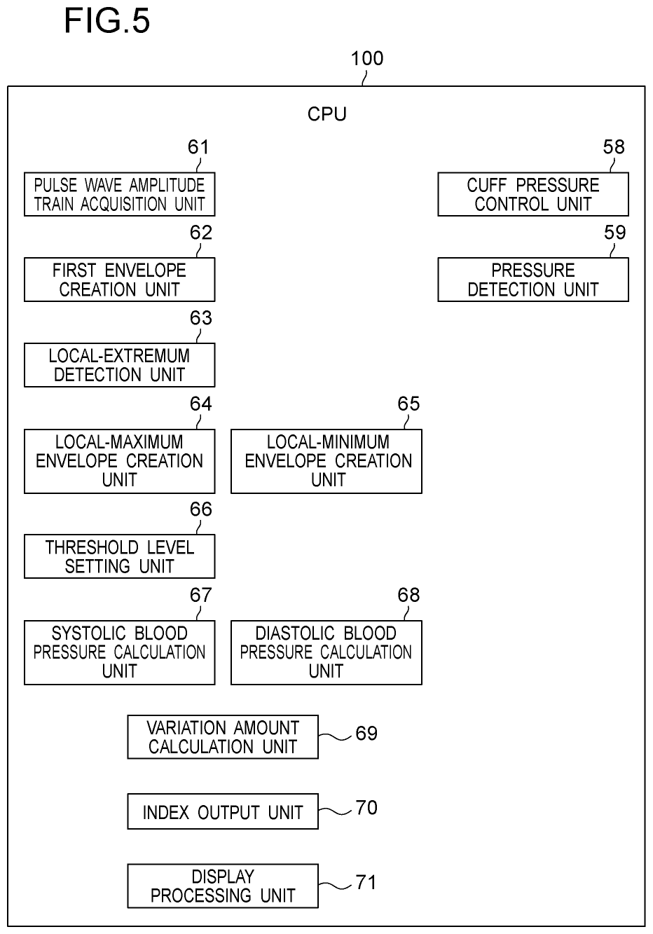

Subsequently, the CPU 100 functions as a cuff pressure control unit 58 (see FIG. 5) to close the valve 33 via the valve drive circuit 330, and then drives the pump 32 via the pump drive circuit 320 to supply air into the fluid bladder 22. With this control, the fluid bladder 22 is inflated, and the cuff pressure is gradually increased (steps S3 and S4).

When the cuff pressure is increased and reaches a predetermined pressure (YES in step S4), the CPU 100 stops the pump 32 via the pump drive circuit 320, and then performs control for gradually opening the valve 33 via the valve drive circuit 330. With this control, the fluid bladder 22 is deflated, and the cuff pressure is gradually decreased (steps S5 and S6).

Here, the predetermined pressure is a pressure sufficiently higher than the systolic blood pressure of the subject (for example, systolic blood pressure+30 mmHg), and it is stored in the memory 51 in advance, or determined by the CPU 100 estimating the systolic blood pressure based on a predetermined calculation formula while the cuff pressure is increased (see, for example, JP 2001-70263 A).

Also, regarding the pressure decrease rate, a target pressure decrease rate that is to be a target is set while the cuff is being inflated, and the CPU 100 controls the degree of opening of the valve 33 so that the pressure decrease rate reaches the target pressure decrease rate (see JP 2001-70263 A mentioned above).

In the pressure decrease process, the CPU 100 functions as a pressure detection unit 59 (see FIG. 5) to detect the pressure of the cuff 20 by the pressure sensor 31 and obtain a cuff pressure signal (denoted by reference sign Pc). Based on the cuff pressure signal Pc, the CPU 100 calculates blood pressure values (systolic blood pressure and diastolic blood pressure) by applying a later-described algorithm using the oscillometric method (step S6). Note that the blood pressure values may be calculated during the pressure increase process instead of during the pressure decrease process.

When the blood pressure values are calculated and determined (YES in step S6), the CPU 100 performs control for immediately opening the valve 33 via the valve drive circuit 330 and discharging the air in the fluid bladder 22 of the cuff 20 (quick discharge) (step S7) in this example.

Next, in step S8, the CPU 100 calculates a heart failure index relatively representing the severity of heart failure by an algorithm described later.

Next, the CPU 100 functions as a display processing unit 71 (see FIG. 5) to display the calculated blood pressure values and heart failure index on the display 50 (step S9). Further, the CPU 100 performs control for storing the blood pressure values and the heart failure index in the memory 51.

When the power switch 52A is then pressed, the blood pressure meter 1 ends the operation.

### (Calculation of Blood Pressure Values and Heart Failure Index)

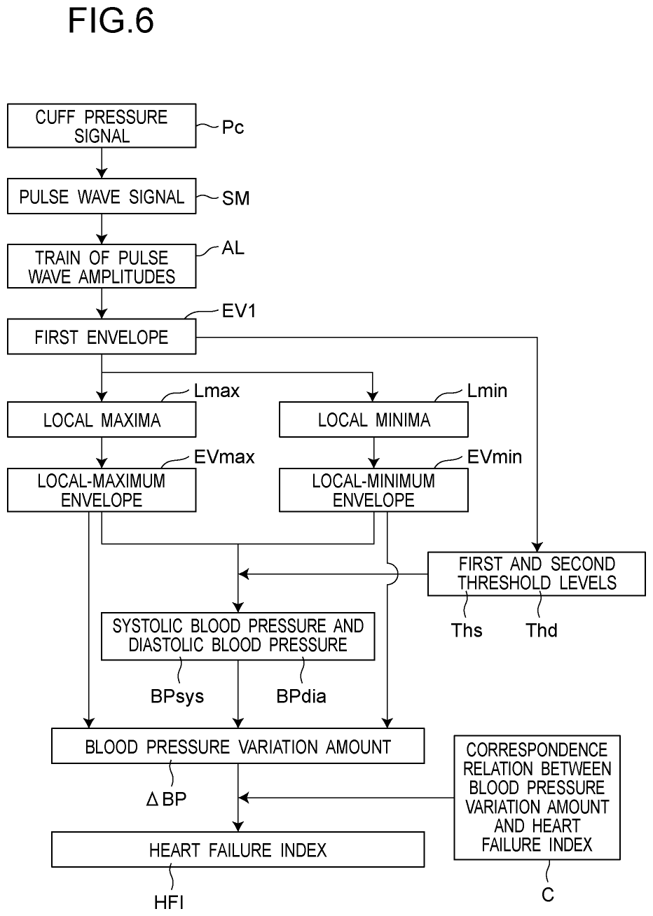

FIG. 5 illustrates elements included in the CPU 100 (software) of the blood pressure meter 1 in order to calculate the blood pressure values and the heart failure index. Note that FIG. 5 shows the cuff pressure control unit 58, the pressure detection unit 59, and the display processing unit 71 described above together with other elements. In this example, the elements for calculating the blood pressure values and the heart failure index include a pulse wave amplitude train acquisition unit 61, a first envelope creation unit 62, a local-extremum detection unit 63, a local-maximum envelope creation unit 64, a local-minimum envelope creation unit 65, a threshold level setting unit 66, a systolic blood pressure calculation unit 67, a diastolic blood pressure calculation unit 68, a variation amount calculation unit 69, and an index output unit 70. FIG. 6 shows a flow of processing when blood pressure values and a heart failure index are calculated by the elements in FIG. 5.

A method for calculating the blood pressure values and the heart failure index based on the cuff pressure signal Pc will be described mainly with reference to FIGS. 5 and 6.

i) First, as shown in FIG. 6, the pulse wave amplitude train acquisition unit 61 shown in FIG. 5 receives the cuff pressure signal Pc detected by the abovementioned pressure sensor 31 and extracts a pulse wave signal SM that is superimposed on the cuff pressure signal Pc and represents a pulse wave at the measurement site.

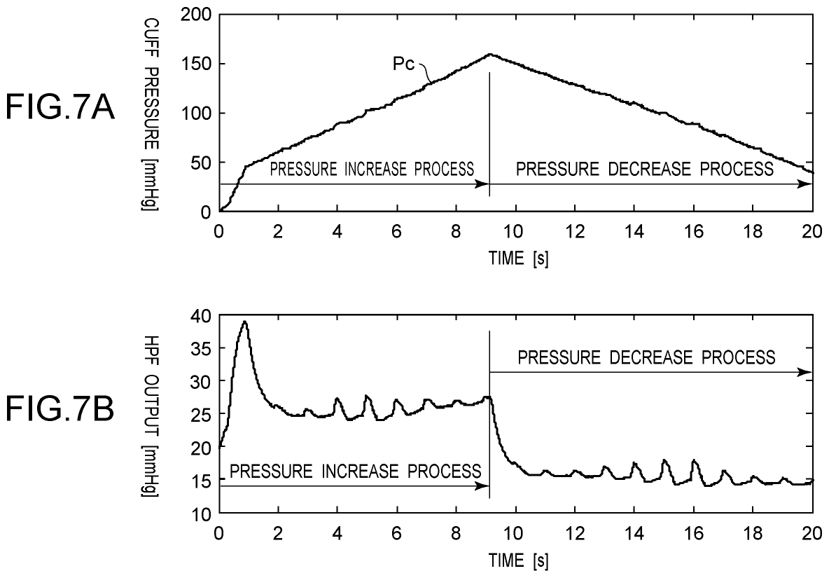

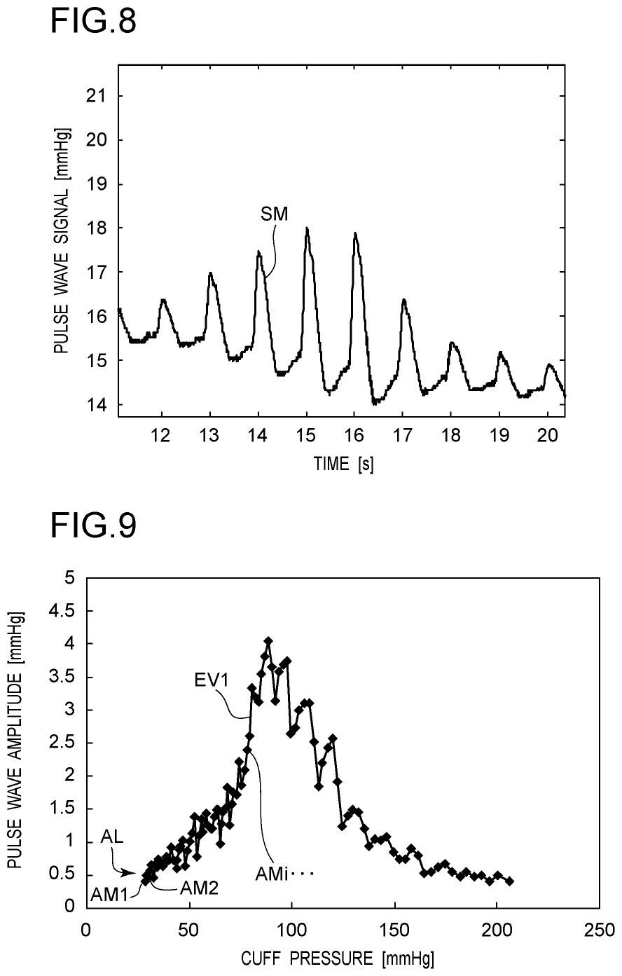

Here, as shown in FIG. 7A, the cuff pressure signal Pc is a signal in which a variation component corresponding to changes in arterial volume for each heartbeat is superimposed on the pressure that rises (pressure increase process) or falls (pressure decrease process) almost linearly over time. The pulse wave amplitude train acquisition unit 61 extracts the variation component (HPF output) as shown in FIG. 7B from the cuff pressure signal Pc via a high-pass filter (HPF), and outputs the extracted component as a pulse wave signal SM as shown in FIG. 8. In this example, as shown in FIG. 8 (corresponding to the pressure decrease process), according to the variation in the arterial volume, the pulse wave signal SM starts to increase about 12 seconds from the start of measurement, reaches its maximum about 16 seconds from the start of measurement, and mostly dissipates about 20 seconds from the start of measurement.

Then, the pulse wave amplitude train acquisition unit 61 acquires a train AL of amplitudes indicated by the pulse wave signal SM (hereinafter referred to as “pulse wave amplitudes” as appropriate). In this example, as shown in FIG. 9, the train AL of pulse wave amplitudes is represented as a train AL of amplitudes (peak values) AM₁, AM₂, . . . , AMᵢ, . . . per beat on a plane where the cuff pressure and the pulse wave amplitude form orthogonal coordinates, with the cuff pressure on the horizontal axis.

ii) Next, as shown in FIG. 6, the first envelope creation unit 62 in FIG. 5 creates, with respect to the train AL of pulse wave amplitudes acquired by the pulse wave amplitude train acquisition unit 61, a first envelope EV1 that connects the amplitudes. Here, the first envelope EV1 has peaks and troughs caused by respiratory variation, as shown in FIG. 9.

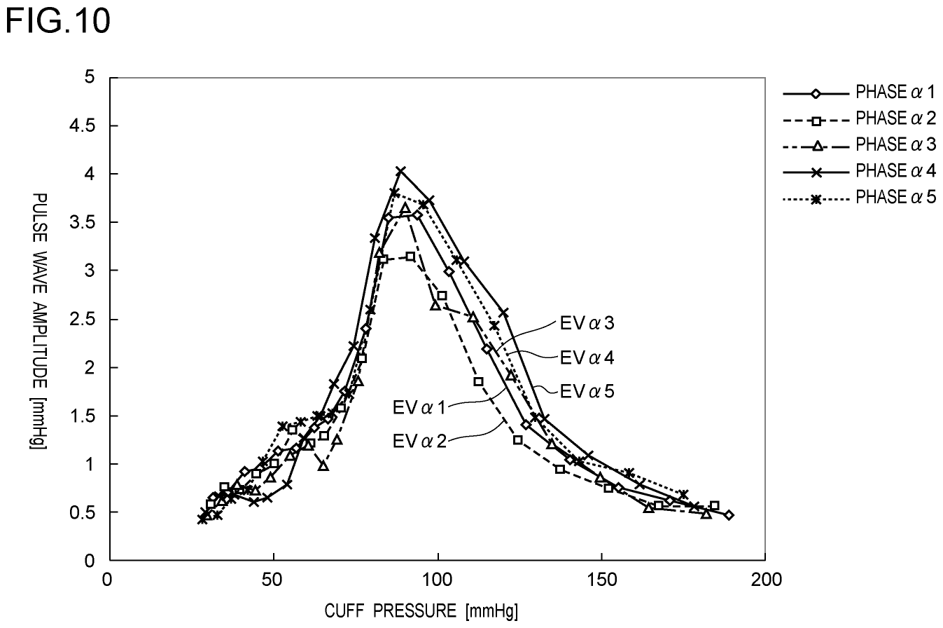

For reference, FIG. 10 shows an example in which, in the case where the respiratory period of the subject is known, a train of amplitudes acquired for each phase α1, α2, . . . , α5 of the respiratory period is acquired from the train AL of pulse wave amplitudes of the pulse wave signal SM, and envelopes EVα1, EVα2, . . . EVα5 are created respectively for the trains of amplitudes for the phases α1, α2, . . . α5. The phases α1, α2, . . . α5 differ by 60° with one breathing cycle being defined as 360°. In the example shown in FIG. 10, EVα5 corresponds to an envelope when the respiratory variation is at local maxima, and EVα2 corresponds to an envelope when the respiratory variation is at local minima. In order to obtain an average blood pressure value, the envelope when such a respiratory variation is at local maxima and the envelope when such a respiration variation is at local minima can be considered as an upper-limit line and a lower-limit line with the respiratory variation taken into account, respectively.

iii) In view of this, as shown in FIG. 6, the local-extremum detection unit 63 in FIG. 5 detects local maxima Lmax and local minima Lmin in the first envelope EV1. Each of the local maxima Lmax and the local minima Lmin forms a train of multiple points.

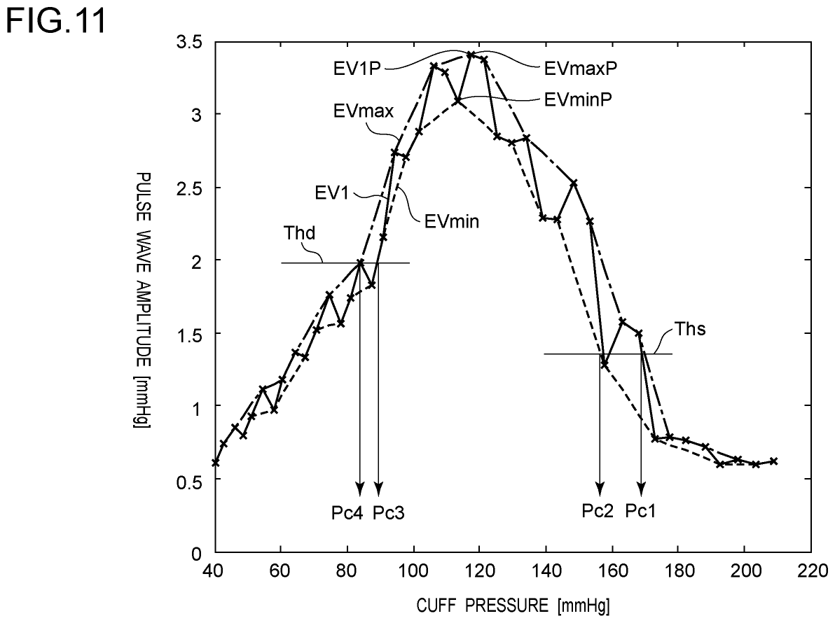

iv) Next, the local-maximum envelope creation unit 64 shown in FIG. 5 creates, with respect to a train of amplitudes corresponding to the local maxima Lmax in the train AL of the pulse wave amplitudes acquired by the pulse wave amplitude train acquisition unit 61, a local-maximum envelope EVmax connecting the amplitudes as shown in FIG. 11. On the other hand, the local-minimum envelope creation unit 65 shown in FIG. 5 creates, with respect to a train of amplitudes corresponding to the local minima Lmin in the train AL of the pulse wave amplitudes acquired by the pulse wave amplitude train acquisition unit 61, a local-minimum envelope EVmin connecting the amplitudes as shown in FIG. 11.

v) Also, in order to obtain the systolic blood pressure BPsys and the diastolic blood pressure BPdia, the threshold level setting unit 66 shown in FIG. 5 calculates and sets a first threshold level Ths and a second threshold level Thd, which are percentages determined in advance with respect to the value of the maximum peak EV1P of the first envelope EV1. In this example, the first threshold level Ths is set to 40% of the value of the maximum peak EV1P, and the second threshold level Thd is set to 50% of the value of the maximum peak EV1P.

vi) Next, the systolic blood pressure calculation unit 67 shown in FIG. 5 obtains two pressure values Pc1 and Pc2 at the points at which the local-maximum envelope EVmax and the local-minimum envelope EVmin cross the first threshold level Ths on the high-pressure side with respect to the maximum peaks EVmaxP and EVminP, as shown in FIGS. 6 and 11. In this example, the systolic blood pressure calculation unit 67 calculates an average value of the two pressure values ((Pc1+Pc2)/2) as the systolic blood pressure BPsys. Further, the diastolic blood pressure calculation unit 68 shown in FIG. 5 obtains two pressure values Pc3 and Pc4 at the points at which the local-maximum envelope EVmax and the local-minimum envelope EVmin cross the second threshold level Thd on the low-pressure side with respect to the maximum peaks EVmaxP and EVminP, as shown in FIGS. 6 and 11. In this example, the diastolic blood pressure calculation unit 68 calculates an average value of the two pressure values ((Pc3+Pc4)/2) as the diastolic blood pressure BPdia.

Here, the local-maximum envelope EVmax and the local-minimum envelope EVmin correspond to the upper-limit line and the lower-limit line of the respiratory variation, respectively. Accordingly, it can be considered that the average value of the two high-pressure-side pressure values ((Pc1+Pc2)/2), and the average value of the two low-pressure-side pressure values ((Pc3+Pc4)/2) are average values with the respiratory variation taken into account, respectively. Thus, according to the electronic blood pressure meter 1, average blood pressure values with the respiratory variation taken into account can be calculated.

vii) Next, as shown in FIG. 6, the variation amount calculation unit 69 in FIG. 5 calculates a blood pressure variation amount ΔBP synchronized with respiration (respiratory variation) based on the local-maximum envelope EVmax, the local-minimum envelope EVmin, and in this example, the systolic blood pressure BPsys.

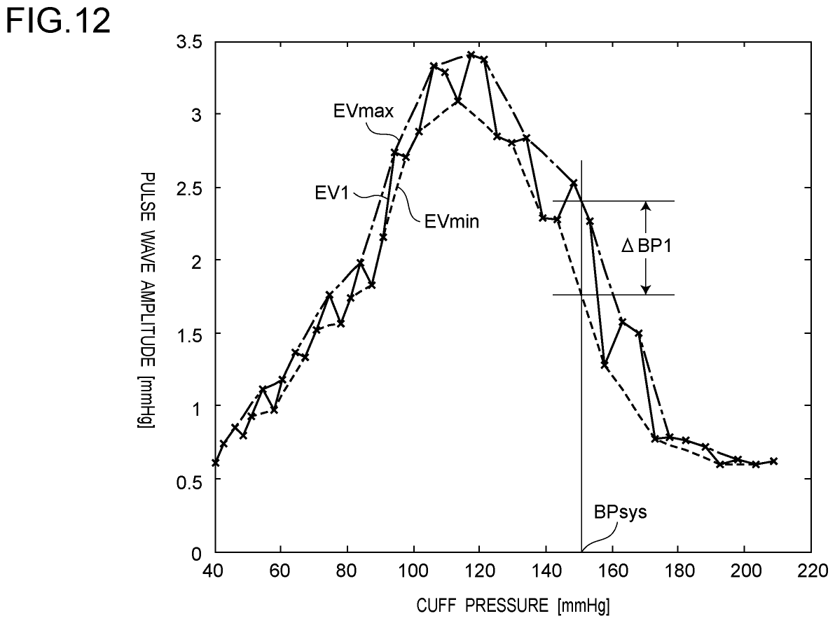

In this example, a blood pressure variation amount ΔBP1 as shown in FIG. 12 is calculated as the blood pressure variation amount ΔBP. The blood pressure variation amount ΔBP1 is defined as a difference (first difference) between the pulse wave amplitude of the local-maximum envelope EVmax at a certain cuff pressure and the pulse wave amplitude of the local-minimum envelope EVmin at the certain cuff pressure. In the example of FIG. 12, the systolic blood pressure BPsys calculated by the systolic blood pressure calculation unit 67 is adopted as the “certain cuff pressure”. As a result, the blood pressure variation amount (respiratory variation) ΔBP synchronized with respiration can be actually obtained.

Note that the pulse wave amplitude varies depending on the manner of wrapping the cuff. For example, when the cuff is loosely wrapped, the pulse wave amplitude is small, and when the cuff is tightly wrapped, the pulse wave amplitude is large. In order to reduce the influence, it is more desirable that, when the local-maximum envelope EVmax and the local-minimum envelope EVmin are obtained, they are created using normalized pulse wave amplitude values obtained by normalizing the pulse wave amplitude values by the maximum value of the first envelope EV1.

viii) As shown in FIG. 6, the index output unit 70 in FIG. 5 outputs a predetermined numerical value corresponding to the blood pressure variation amount ΔBP as a heart failure index HFI representing the relative severity of heart failure. Specifically, the index output unit 70 obtains the heart failure index HFI corresponding to the blood pressure variation amount ΔBP (in this example, the blood pressure variation amount ΔBP1 shown in FIG. 12) by referring to the correspondence relation C, stored in advance in the memory 51, between the blood pressure variation amount and the heart failure index shown in FIG. 3. Thus, the heart failure index HFI can be output smoothly.

Specifically, in this example, the correspondence relation C between the blood pressure variation amount and the heart failure index is stored in the memory 51 in the form of a linear function y=33x−0.67. Here, the variable x represents the blood pressure variation amount ΔBP, and the variable y represents the heart failure index HFI. In this example, the heart failure index HFI is represented by a single-digit number from 1 to 5, rounded to the nearest whole number. In particular, if the blood pressure variation amount ΔBP is less than 0.01 [mmHg], the heart failure index HFI is rounded up to 1. If the blood pressure variation amount ΔBP exceeds 0.17 [mmHg], the heart failure index HFI is rounded down to 5. As described above, if the heart failure index HFI is represented by a single-digit number, general users can easily know the severity of heart failure.

ix) The display processing unit 71 in FIG. 5 performs processing of displaying the obtained heart failure index HFI on the display 50 together with the calculated blood pressure values (systolic blood pressure BPsys and diastolic blood pressure BPdia).

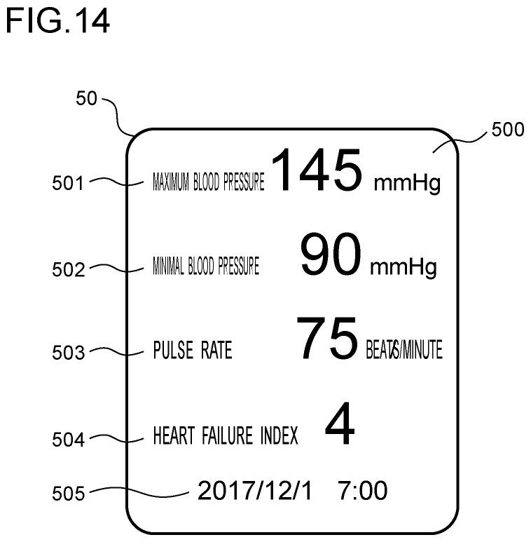

In this example, as shown in FIG. 14, a display screen 500 of the display 50 includes, in order from the top, a “maximum blood pressure” display area 501 that displays a value of the systolic blood pressure BPsys, a “minimal blood pressure” display area 502 that displays a value of the diastolic blood pressure BPdia, a “pulse rate” display area 503 that displays pulse rate, a “heart failure index” display area 504 that displays a value of the heart failure index HFI, and a measurement date/time display area 505 that displays a measurement date and time. In the example of FIG. 14, 145 mmHg is displayed in the “maximum blood pressure” display area 501, 90 mmHg is displayed in the “minimal blood pressure” display area 502, 75 beats/minute is displayed in the “pulse rate” display area 503, “4” is displayed in the “heart failure index” display area 504 as the value of the heart failure index, and “2017/12/1 7:00” is displayed in the measurement date/time display area 505 as the measurement date and time.

In this example, the magnitude of the numerical value of the heart failure index HFI corresponds to the level of severity of heart failure. The user can find an extent to which the condition of heart failure deteriorates by seeing the numerical value of the heart failure index displayed in the “heart failure index” display area 504. If the condition of heart failure has deteriorated to some extent, appropriate measures can be taken, such as visiting a hospital to see a doctor even on a day other than the scheduled consultation day.

Here, the cuff pressure control unit 58 using the pump 32 and the valve 33, and the pressure detection unit 59 using the pressure sensor 31 are components included in a popular commercially available electronic blood pressure meter for obtaining the blood pressure at the measurement site with the oscillometric method, and those components do not require invasion to the subject. Further, the elements 61 to 71 included in the CPU 100 shown in FIG. 5 each perform calculations using the cuff pressure signal Pc and the blood pressure variation amount ΔBP (amount obtained based on the cuff pressure signal Pc), and do not require invasion to the subject. Therefore, the blood pressure meter 1 can output the heart failure index HFI non-invasively to the subject.

### (Verification of Validity of Heart Failure Index)

In order to verify the validity of the abovementioned heart failure index HFI, the present inventor measured the heart failure indexes HFI obtained by the blood pressure meter 1 and NT-proBNP (N-terminal pro B-type natriuretic peptide), which is one of the biomarkers indicating the severity of heart failure, for two heart failure patients (patient A and patient B), on the date of admission and the date of discharge. Here, NT-proBNP is used as a biomarker indicating the severity of heart failure because the numerical value (concentration in blood) increases as the heart function decreases and the heart load increases. Specifically, it is generally said that, when NT-proBNP is 125 (pg/ml) or more, the patient may have a mild heart failure, and when NT-proBNP is 900 (pg/ml) or more, the patient may have a heart failure that requires treatment. However, NT-proBNP may show a high value (level) due to factors other than heart failure, such as a decrease in renal function, so that it may vary considerably by individual.

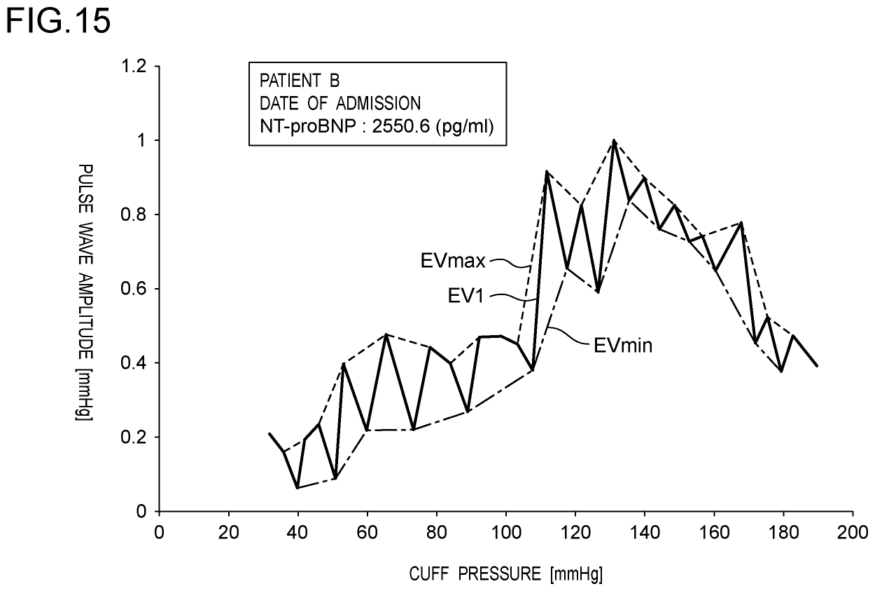

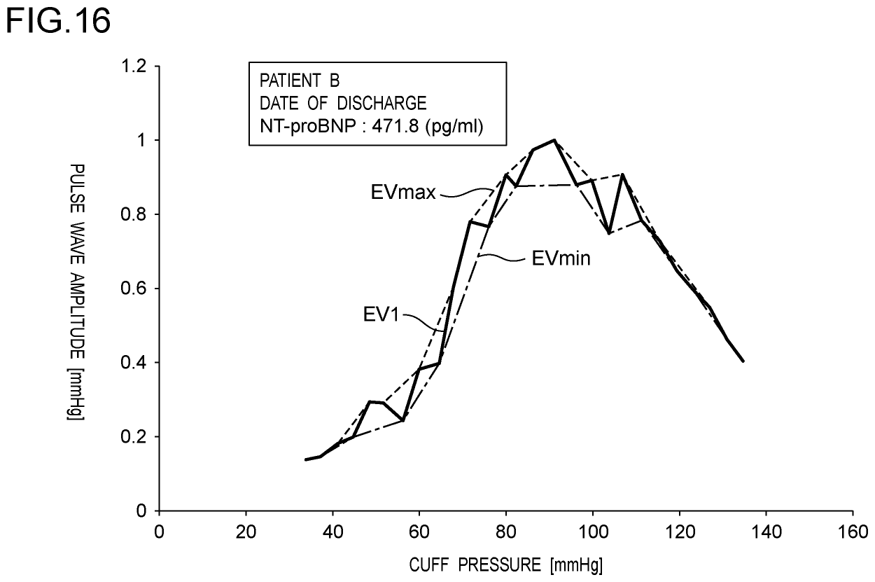

FIGS. 15 and 16 show a local-maximum envelope EVmax and a local-minimum envelope EVmin created for, for example, the patient B by the blood pressure meter 1 on the date of admission and the date of discharge, respectively. On the date of admission, NT-proBNP was 2550.6 [pg/ml], and the severity of heart failure was relatively high, as shown in FIG. 15. At this time, the difference between the local-maximum envelope EVmax and the local-minimum envelope EVmin was relatively large, and the blood pressure variation amount ΔBP was also relatively large. On the date of discharge, NT-proBNP was 471.8 [pg/ml], and the severity of heart failure was relatively lowered, as shown in FIG. 16. At this time, the difference between the local-maximum envelope EVmax and the local-minimum envelope EVmin was relatively small, and the blood pressure variation amount ΔBP was also relatively small. The similar tendency was observed for the patient A.

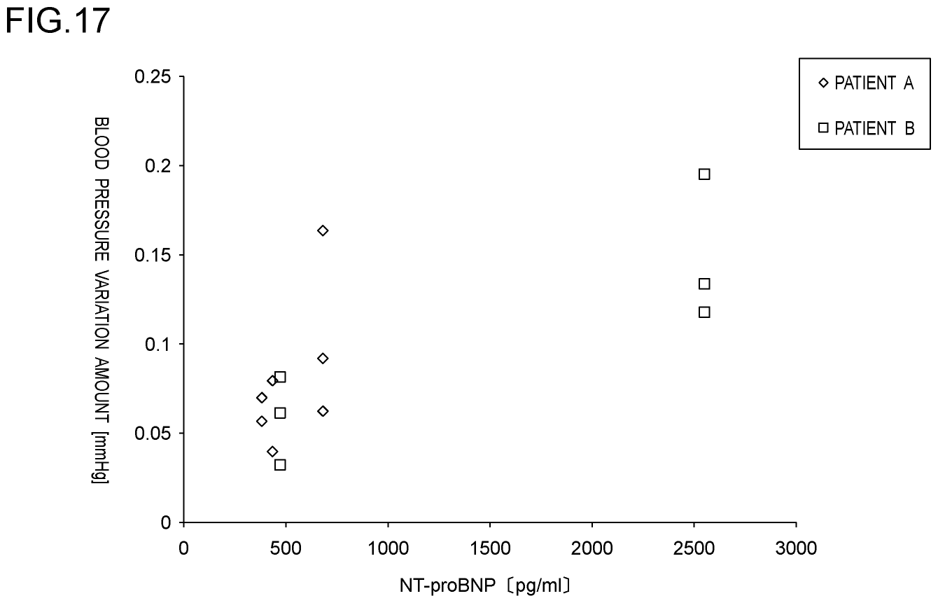

FIG. 17 is a scatter diagram showing the relation between the blood pressure variation amount ΔBP (in this example, ΔBP1 shown in FIG. 12) obtained by the blood pressure meter 1 and NT-proBNP for each of the patients A and B on the date of admission and on the date of discharge. In FIG. 17, the mark □ shows the data of the patient A and the mark ⋄ shows the data of the patient B. It can be seen from FIG. 17 that the blood pressure variation amount ΔBP increases with an increase in NT-proBNP for both the patients A and B.

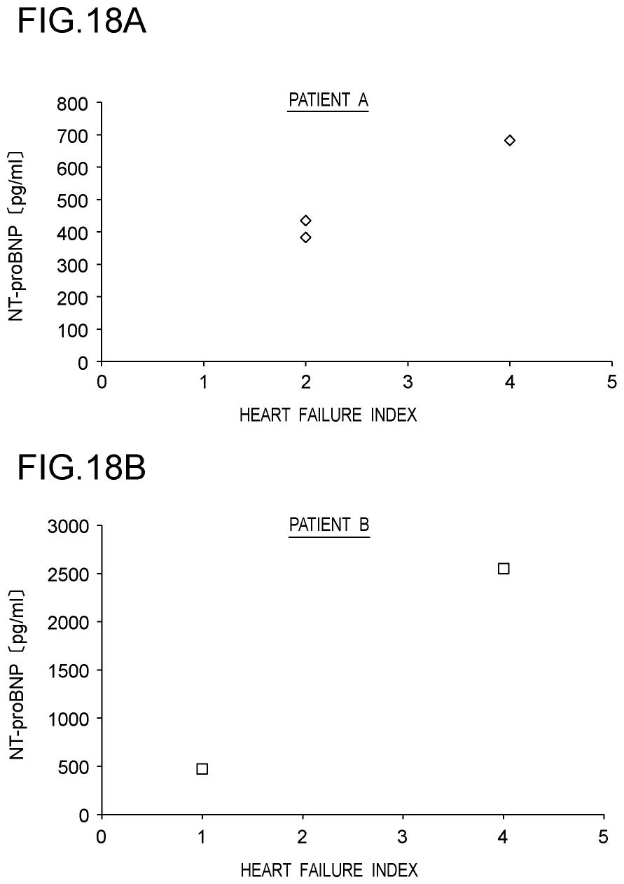

FIG. 18A is a scatter diagram showing the relation between the heart failure index HFI obtained by the blood pressure meter 1 and NT-proBNP, those of which were measured on the date of admission and the date of discharge, for the patient A. Similarly, FIG. 18B is a scatter diagram showing the relation between the heart failure index HFI obtained by the blood pressure meter 1 and NT-proBNP, those of which were measured on the date of admission and the date of discharge, for the patient B. As can be seen from FIGS. 18A and 18B, the magnitude of the heart failure index HFI obtained by the blood pressure meter 1 corresponds to the magnitude of NT-proBNP for both the patient A and patient B. Therefore, it can be considered that the heart failure index HFI obtained by the blood pressure meter 1 represents the relative severity of heart failure.

In FIGS. 18A and 18B, the scales of NT-proBNP values are relatively greatly different, which is considered to be due to individual differences as described above. Considering the value of NT-proBNP on the date of admission and on the date of discharge, the patient A is recommended to take an action such as visiting a hospital to see a doctor, when the heart failure index HFI by the blood pressure meter 1 becomes, for example, 3 or more. Considering the value of NT-proBNP on the date of admission and on the date of discharge, the patient B is recommended to take an action such as visiting a hospital to see a doctor, when the heart failure index HFI by the blood pressure meter 1 becomes, for example, 2 or 3 or more. As described above, the blood pressure meter 1 can be used for home-based monitoring and/or screening for heart failure.

In addition, if the heart failure index HFI by the blood pressure meter 1 is equal to or higher than a predetermined threshold (for example, 3 or more as a value indicating that medical attention is needed), the CPU 100 may function as an alarm unit, in addition to simply display the numerical value of the heart failure index HFI on the display 50. For example, the CPU 100 may notify the user of such situation by, for example, blinking the numerical value of the heart failure index HFI in the display screen 500 or issuing an alarm sound with the buzzer 55. This clearly prompts the user to see a doctor. Further, it is desirable that the threshold for the CPU 100 to function as the alarm unit can be variably set by operating the operation unit 52, for example. Thus, the threshold can be appropriately set according to each subject (patient).

### (Another Definition of Blood Pressure Variation Amount)

In the above example, the blood pressure variation amount synchronized with respiration (respiratory variation) ΔBP is the blood pressure variation amount ΔBP1 shown in FIG. 12, that is, a difference (first difference) between the pulse wave amplitude of the local-maximum envelope EVmax and the pulse wave amplitude of the local-minimum envelope EVmin at a certain cuff pressure (in the above example, systolic blood pressure BPsys). However, it is not limited thereto. The blood pressure variation amount ΔBP may be a ratio (first ratio) between a pulse wave amplitude of the local-maximum envelope EVmax and a pulse wave amplitude of the local-minimum envelope EVmin at a certain cuff pressure.

Further, as the “certain cuff pressure” that gives the first difference or ratio, the diastolic blood pressure BPdia calculated by the diastolic blood pressure calculation unit 68 may be used instead of the systolic blood pressure BPsys, or a pressure value obtained by adding a predetermined constant value (for example, 10 mmHg) to the cuff pressure at which the local-maximum envelope EVmax or the local-minimum envelope EVmin has the maximum peak EVmaxP or EVminP may be used.

Note that there may be a plurality of the “certain cuff pressures” that gives the first difference or ratio. In that case, it is desirable that statistical processing (e.g., processing to obtain an average value) is performed on the first differences or ratios obtained according to the plurality of cuff pressures, and the resultant value is set as the blood pressure variation amount ΔBP.

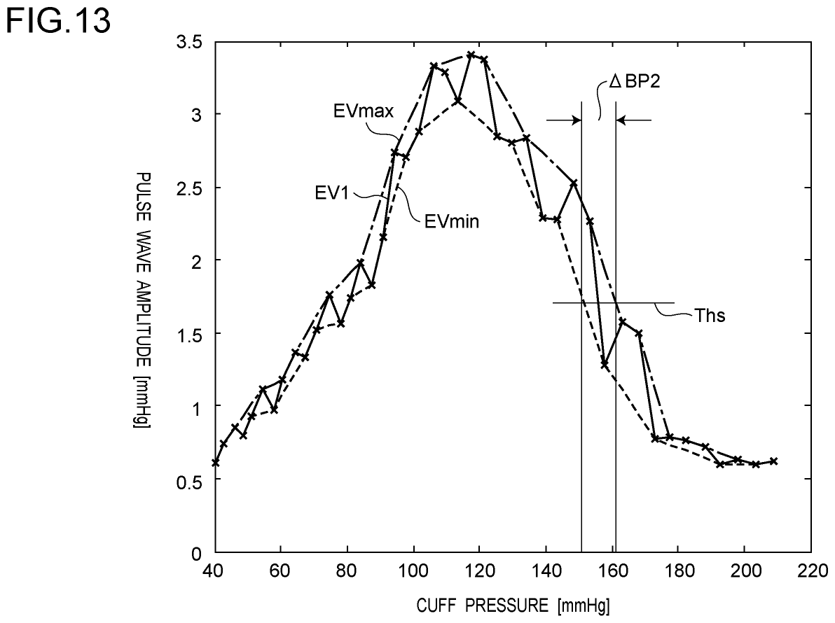

Instead of the first difference or ratio, the blood pressure variation amount (respiratory variation) ΔBP synchronized with respiration may be a blood pressure variation amount ΔBP2 shown in FIG. 13, that is, a difference (second difference) between a cuff pressure Pei at which the local-maximum envelope EVmax has a certain pulse wave amplitude (in this example, the first threshold level Ths) and a cuff pressure Pc2 at which the local-minimum envelope EVmin has the certain pulse wave amplitude on the high-pressure side with respect to the maximum peaks EVmaxP and EVminP of the local-maximum envelope EVmax and the local-minimum envelope EVmin shown in FIG. 11. Alternatively, the blood pressure variation amount (respiratory variation) ΔBP synchronized with respiration may be a ratio (second ratio) between a cuff pressure Pc1 at which the local-maximum envelope EVmax has a certain pulse wave amplitude (for example, the first threshold level Ths) and a cuff pressure Pc2 at which the local-minimum envelope EVmin has the certain pulse wave amplitude on the high-pressure side with respect to the maximum peaks EVmaxP and EVminP of the local-maximum envelope EVmax and the local-minimum envelope EVmin. Alternatively, the blood pressure variation amount (respiratory variation) ΔBP synchronized with respiration may be a difference or ratio between a cuff pressure Pc3 at which the local-maximum envelope EVmax has a certain pulse wave amplitude (for example, the second threshold level Thd) and a cuff pressure Pc4 at which the local-minimum envelope EVmin has the certain pulse wave amplitude on the low-pressure side with respect to the maximum peaks EvmaxP and EVminP of the local-maximum envelope EVmax and the local-minimum envelope EVmin.

Further, the “certain pulse wave amplitude” that gives the second difference or ratio may be changed with respect to the first threshold level Ths and the second threshold level Thd on the high-pressure side and the low-pressure side with respect to the maximum peak EVmaxP of the local-maximum envelope EVmax and the maximum peak EVminP of the local-minimum envelope EVmin.

Note that there may be a plurality of the “certain pulse wave amplitudes” that gives the second difference or ratio. In that case, it is desirable that statistical processing (e.g., processing to obtain an average value) is performed on the second differences or ratios obtained according to the plurality of pulse wave amplitudes, and the resultant value is set as the blood pressure variation amount ΔBP.

As described above, the blood pressure variation amount ΔBP can be determined in various ways based on the deviation (respiratory variation) between the local-maximum envelope EVmax and the local-minimum envelope EVmin. In that case, it is desirable that the correspondence relation C (FIG. 3) between the blood pressure variation amount and the heart failure index is reset according to the definition of the blood pressure variation amount.

### (Modifications)

In the above example, the correspondence relation C between the blood pressure variation amount and the heart failure index is stored in the form of a function in the memory 51 serving as the correspondence relation storage unit as shown in FIG. 3. However, it is not limited thereto. The correspondence relation C between the blood pressure variation amount and the heart failure index may be stored in various forms such as a correspondence table.

Further, in the above example, the heart failure index HFI is represented by a single-digit number from 1 to 5, rounded to the nearest whole number. However, it is not limited thereto. The heart failure index HFI may be represented by, for example, a single-digit number from 1 to 9 or a multi-digit number.

In the above example, the value of the heart failure index HFI increases as the severity of heart failure increases. However, it is not limited thereto. For example, the value of the heart failure index HFI may be decreased as the severity of heart failure increases by setting the correspondence relation C between the blood pressure variation amount and the heart failure index to have a negative slope in FIG. 3.

In the embodiment described above, the measurement site is the upper arm 90, but the measurement site is not limited thereto. The measurement site may be a wrist or a leg. Further, the main body 10 of the blood pressure meter 1 and the cuff 20 may be integrated.

Further, while the above embodiment describes the electronic blood pressure meter (blood pressure meter 1), the present invention is not limited thereto. The present invention may be embodied as a heart failure detector rather than an electronic blood pressure meter. For example, the heart failure detector has the same appearance (see FIG. 1) and the same block configuration (see FIG. 2) as those of the blood pressure meter 1, and executes processing same as the processing for calculating the heart failure index shown in FIG. 6, non-invasively to the subject. Upon output, the heart failure detector displays, for example, only information regarding the heart failure index (“heart failure index 4” in the example of FIG. 14) on the display screen 500 of the display 50 shown in FIG. 14. The user can find an extent to which the condition of heart failure deteriorates by the heart failure index. If the condition of heart failure has deteriorated to some extent, appropriate measures can be taken, such as visiting a hospital to see a doctor even on a day other than the scheduled consultation day. As described above, the present invention can be embodied as various devices.

The present invention was conceived in the following manner. First, the present inventor has focused on a probability of correlation between a blood pressure variation synchronized with respiration (respiratory variation) and severity of heart failure.

That is, Non-Patent Literature 2 (Azriel Perel et al., “Systolic Blood Pressure Variation is a Sensitive Indicator of Hypovolemia in Ventilated Dogs Subjected to Graded Hemorrhage”, Anesthesiology, 67, PP. 498-502, 1987) reports that systolic pressure variation (SPV) and its Adown component are accurate indicators of hypovolemia in ventilated dogs subjected to hemorrhage. In Non-Patent Literature 2, systolic pressure variation (SPV) is defined as a difference between the maximum and minimum values of systolic blood pressure following a single positive pressure breath. Therefore, the systolic pressure variation (SPV) in Non-Patent Literature 2 is considered to correspond to the “respiratory variation” of blood pressure in the present specification. In this Literature, systolic pressure variation (SPV), that is, the difference between the maximum and minimum values of systolic blood pressure, is, using systolic blood pressure during 5-second apnea period as a reference value, further divided into Δup component higher than the reference value and Adown component lower than the reference value. In addition, heart failure means a condition in which as the function of the heart as a pump deteriorates, and therefore it is impossible to pump sufficient blood to the whole body or to receive sufficient blood that has circulated throughout the whole body. In view of this, the hypovolemia in the above Literature is considered to correspond to the severity of heart failure.

Further, as disclosed in, for example, Patent Literature 1 (JP 2015-9044 A), an electronic blood pressure meter has been proposed which obtains a blood pressure variation amount synchronized with respiration (respiratory variation) and calculates average blood pressure values (systolic blood pressure and diastolic blood pressure) taking the respiratory variation into account, when measuring a blood pressure non-invasively according to the oscillometric method.

In view of this, the present inventor has conceived of obtaining a blood pressure variation amount synchronized with respiration (respiratory variation) using an electronic blood pressure meter that measures the blood pressure at a measurement site according to an oscillometric method, and obtaining a heart failure index relatively representing severity of heart failure based on the obtained blood pressure variation amount.

As described above, an electronic blood pressure meter according to the present disclosure is an electronic blood pressure meter for measuring a blood pressure at a measurement site using an oscillometric method, the electronic blood pressure meter comprising:

a cuff pressure control unit capable of changing a pressure of a cuff worn on the measurement site;

a pressure detection unit that detects a cuff pressure signal representing the pressure of the cuff;

a variation amount calculation unit that obtains a blood pressure variation amount synchronized with respiration based on the cuff pressure signal;

a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and

an index output unit that refers to the correspondence relation stored in the correspondence relation storage unit and outputs the heart failure index corresponding to the blood pressure variation amount obtained by the variation amount calculation unit.

In the present specification, the wording “the heart failure index representing ‘relative’ severity of heart failure” means that the magnitude of the numerical value of the heart failure index corresponds to the level of severity of heart failure (means that, as the numerical value of the heart failure index increases, the level of severity of heart failure increases (or may decrease)).

In the electronic blood pressure meter according to the present disclosure, the cuff pressure control unit changes the pressure of the cuff worn on the measurement site during measurement. In the process of decreasing or increasing the pressure of the cuff, the pressure detection unit detects a cuff pressure signal representing the pressure of the cuff. Based on the cuff pressure signal, the blood pressure (systolic blood pressure and diastolic blood pressure) at the measurement site is obtained using the oscillometric method. Further, in this electronic blood pressure meter, the variation amount calculation unit obtains, based on the cuff pressure signal, a blood pressure variation amount synchronized with respiration. Herein, the correspondence relation storage unit that stores a predetermined correspondence relation between a blood pressure variation amount synchronized with respiration and a heart failure index that numerically represents relative severity of heart failure and corresponds to a value of a biomarker indicating severity of heart failure. The index output unit refers to the correspondence relation stored in the correspondence relation storage unit and outputs the heart failure index corresponding to the blood pressure variation amount obtained by the variation amount calculation unit.

Here, the cuff pressure control unit and the pressure detection unit are components included in a popular commercially available electronic blood pressure meter for obtaining the blood pressure at the measurement site using the oscillometric method, and those components do not require invasion to the subject. Further, the variation amount calculation unit and the index output unit are components that perform calculations using the cuff pressure signal and the blood pressure variation amount (amount obtained based on the cuff pressure signal), respectively, and do not require invasion to the subject. Accordingly, this electronic blood pressure meter can output the above heart failure index non-invasively to the subject. The outputted heart failure index corresponds to the value of the biomarker, and thus, can correctly represent the severity of heart failure. A user (including a subject and those who take care of the subject; the same applies hereafter) can find an extent to which the condition of heart failure deteriorates by the heart failure index. If the condition of heart failure has deteriorated to some extent, appropriate measures can be taken, such as visiting a hospital to see a doctor even on a day other than the scheduled consultation day.

The electronic blood pressure meter according to one embodiment further comprises:

an operation unit capable of variably setting a threshold for the heart failure index; and

an alarm unit that provides a notification indicating that medical attention is needed, according to whether the heart failure index output by the index output unit is greater or smaller than the threshold set by the operation unit.

NT-proBNP (N-terminal pro B-type natriuretic peptide) which is one of biomarkers indicating severity of heart failure may show a high value (level) due to factors other than heart failure, such as a decrease in renal function, so that it may vary considerably by individual. Therefore, the electronic blood pressure meter according to one embodiment is provided with an operation unit capable of variably setting a threshold for the heart failure index. Accordingly, the threshold can be set, as appropriate, according to a level of the biomarker of each subject (patient), for example. The alarm unit notifies that medical attention is needed according to whether the heart failure index output by the index output unit is larger or smaller than the threshold set by the operation unit. Accordingly, a notification indicating that medical attention is needed is provided, as appropriate, according to the level of the biomarker of each subject.

Note that the predetermined correspondence relation between the blood pressure variation amount and the heart failure index can be stored in various forms such as a function and a correspondence table.

The electronic blood pressure meter according to one embodiment further comprises:

a pulse wave amplitude train acquisition unit that extracts a pulse wave signal superimposed on the cuff pressure signal and indicating a pulse wave at the measurement site, and acquires a train of amplitudes indicated by the pulse wave signal;

a first envelope creation unit that creates, with respect to the train of amplitudes acquired by the pulse wave amplitude train acquisition unit, a first envelope connecting the amplitudes;

a local-extremum detection unit that detects local maxima and local minima in the first envelope;

a local-maximum envelope creation unit that creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates; and

a local-minimum envelope creation unit that creates, with respect to a train of amplitudes corresponding to the local minima in the train of amplitudes acquired by the pulse wave amplitude train acquisition unit, a local-minimum envelope connecting the amplitudes on the plane,

wherein the variation amount calculation unit obtains, as the blood pressure variation amount, a first difference or ratio between a pulse wave amplitude of the local-maximum envelope at a certain cuff pressure and a pulse wave amplitude of the local-minimum envelope at the certain cuff pressure on the plane.

Here, the first envelope, the local-maximum envelope, and the local-minimum envelope are typically represented on a plane with the horizontal axis representing a cuff pressure and the vertical axis representing a pulse wave amplitude.

In the electronic blood pressure meter according to one embodiment, the pulse wave amplitude train acquisition unit extracts a pulse wave signal that is superimposed on the cuff pressure signal and that indicates a pulse wave at the measurement site, and acquires a train of amplitudes indicated by the pulse wave signal. The first envelope creation unit creates, with respect to the train of amplitudes acquired by the pulse wave amplitude train acquisition unit, a first envelope connecting the amplitudes. The local-extremum detection unit detects local maxima and local minima in the first envelope. The local-maximum envelope creation unit creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates. The local-minimum envelope creation unit creates, with respect to a train of amplitudes corresponding to the local minima in the train of amplitudes acquired by the pulse wave amplitude train acquisition unit, a local-minimum envelope connecting the amplitudes on the plane. The variation amount calculation unit obtains, as the blood pressure variation amount, a first difference or ratio between a pulse wave amplitude of the local-maximum envelope at a certain cuff pressure and a pulse wave amplitude of the local-minimum envelope at the certain cuff pressure on the plane. As a result, the blood pressure variation amount synchronized with respiration can be actually obtained.

Note that there may be a plurality of the “certain cuff pressures” that gives the first difference or ratio. In that case, it is desirable that statistical processing (e.g., processing to obtain an average value) is performed on the first differences or ratios obtained according to the plurality of cuff pressures, and the resultant value is set as the blood pressure variation amount.

The electronic blood pressure meter according to one embodiment further comprises:

a pulse wave amplitude train acquisition unit that extracts a pulse wave signal superimposed on the cuff pressure signal and indicating a pulse wave at the measurement site, and acquires a train of amplitudes indicated by the pulse wave signal;

a first envelope creation unit that creates, with respect to the train of amplitudes acquired by the pulse wave amplitude train acquisition unit, a first envelope connecting the amplitudes;

a local-extremum detection unit that detects local maxima and local minima in the first envelope;

a local-maximum envelope creation unit that creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates; and

a local-minimum envelope creation unit that creates, with respect to a train of amplitudes corresponding to the local minima in the train of amplitudes acquired by the pulse wave amplitude train acquisition unit, a local-minimum envelope connecting the amplitudes on the plane,

wherein the variation amount calculation unit obtains, as the blood pressure variation amount, a second difference or ratio between a cuff pressure at which the local-maximum envelope has a certain pulse wave amplitude and a cuff pressure at which the local-minimum envelope has the certain pulse wave amplitude on a high-pressure side with respect to maximum peaks of the local-maximum envelope and the local-minimum envelope or on a low-pressure side with respect to the maximum peaks of the local-maximum envelope and the local-minimum envelope on the plane.

Here, as is the case described previously, the first envelope, the local-maximum envelope, and the local-minimum envelope are typically represented on a graph with the horizontal axis representing a cuff pressure and the vertical axis representing a pulse wave amplitude.

In the electronic blood pressure meter according to one embodiment, the pulse wave amplitude train acquisition unit extracts a pulse wave signal that is superimposed on the cuff pressure signal and that indicates a pulse wave at the measurement site, and acquires a train of amplitudes indicated by the pulse wave signal. The first envelope creation unit creates, with respect to the train of amplitudes acquired by the pulse wave amplitude train acquisition unit, a first envelope connecting the amplitudes. The local-extremum detection unit detects local maxima and local minima in the first envelope. The local-maximum envelope creation unit creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates. The local-minimum envelope creation unit creates, with respect to a train of amplitudes corresponding to the local minima in the train of amplitudes acquired by the pulse wave amplitude train acquisition unit, a local-minimum envelope connecting the amplitudes on the plane. The variation amount calculation unit obtains, as the blood pressure variation amount, a second difference or ratio between a cuff pressure at which the local-maximum envelope has a certain pulse wave amplitude and a cuff pressure at which the local-minimum envelope has the certain pulse wave amplitude on a high-pressure side with respect to maximum peaks of the local-maximum envelope and the local-minimum envelope or on a low-pressure side with respect to the maximum peaks of the local-maximum envelope and the local-minimum envelope on the plane. As a result, the blood pressure variation amount synchronized with respiration can be actually obtained.

Note that there may be a plurality of the “certain pulse wave amplitudes” that gives the second difference or ratio. In that case, it is desirable that statistical processing (e.g., processing to obtain an average value) is performed on the second differences or ratios obtained according to the plurality of pulse wave amplitudes, and the resultant value is set as the blood pressure variation amount.

The electronic blood pressure meter according to one embodiment further comprises:

a display; and

a display processing unit that performs processing of displaying the heart failure index on the display together with a calculation result of blood pressure according to the oscillometric method.

In the electronic blood pressure meter according to one embodiment, the heart failure index is displayed on the display together with a calculation result of blood pressure with the oscillometric method. A user (including a subject and those who take care of the subject; the same applies hereafter) can find the extent to which the condition of heart failure deteriorates by seeing the displayed heart failure index along with the calculation result of blood pressure with the oscillometric method.

In another aspect, a heart failure detector of the present disclosure is a heart failure detector for outputting an index relatively representing severity of heart failure, the heart failure detector comprising:

a cuff pressure control unit capable of changing a pressure of a cuff worn on a measurement site;

a pressure detection unit that detects a cuff pressure signal representing the pressure of the cuff;

a variation amount calculation unit that obtains a blood pressure variation amount synchronized with respiration based on the cuff pressure signal;

a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and

an index output unit that refers to the correspondence relation stored in the correspondence relation storage unit and outputs the heart failure index corresponding to the blood pressure variation amount obtained by the variation amount calculation unit.

Similar to the electronic blood pressure meter according to the aspect described previously, the heart failure detector according to the present disclosure can output the heart failure index non-invasively to the subject. The outputted heart failure index corresponds to the value of the biomarker, and thus, can correctly represent the severity of heart failure. The user can find an extent to which the condition of heart failure deteriorates by the heart failure index. If the condition of heart failure has deteriorated to some extent, appropriate measures can be taken, such as visiting a hospital to see a doctor even on a day other than the scheduled consultation day.

As is apparent from the above, the electronic blood pressure meter according to the present disclosure is an electronic blood pressure meter that measures the blood pressure at a measurement site with the oscillometric method and can output a heart failure index representing the relative severity of heart failure non-invasively to the subject. Further, similar to the electronic blood pressure meter described above, the heart failure detector according to the present disclosure can output the heart failure index non-invasively to the subject.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. An electronic blood pressure meter for measuring a blood pressure at a measurement site using an oscillometric method, the electronic blood pressure meter comprising:
a cuff pressure control unit capable of changing a pressure of a cuff worn on the measurement site;
a pressure detection unit that detects a cuff pressure signal representing the pressure of the cuff;
a variation amount calculation unit that obtains a blood pressure variation amount synchronized with respiration based on the cuff pressure signal;
a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and
an index output unit that refers to the correspondence relation stored in the correspondence relation storage unit and outputs the heart failure index corresponding to the blood pressure variation amount obtained by the variation amount calculation unit.

2. The electronic blood pressure meter according to claim 1, further comprising:
an operation unit capable of variably setting a threshold for the heart failure index; and
an alarm unit that provides a notification indicating that medical attention is needed, according to whether the heart failure index output by the index output unit is greater or smaller than the threshold set by the operation unit.

3. The electronic blood pressure meter according to claim 1, further comprising:
a pulse wave amplitude train acquisition unit that extracts a pulse wave signal superimposed on the cuff pressure signal and indicating a pulse wave at the measurement site, and acquires a train of amplitudes indicated by the pulse wave signal;
a first envelope creation unit that creates, with respect to the train of amplitudes acquired by the pulse wave amplitude train acquisition unit, a first envelope connecting the amplitudes;
a local-extremum detection unit that detects local maxima and local minima in the first envelope;
a local-maximum envelope creation unit that creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates; and
a local-minimum envelope creation unit that creates, with respect to a train of amplitudes corresponding to the local minima in the train of amplitudes acquired by the pulse wave amplitude train acquisition unit, a local-minimum envelope connecting the amplitudes on the plane,
wherein the variation amount calculation unit obtains, as the blood pressure variation amount, a first difference or ratio between a pulse wave amplitude of the local-maximum envelope at a certain cuff pressure and a pulse wave amplitude of the local-minimum envelope at the certain cuff pressure on the plane.

4. The electronic blood pressure meter according to claim 1, further comprising:
a pulse wave amplitude train acquisition unit that extracts a pulse wave signal superimposed on the cuff pressure signal and indicating a pulse wave at the measurement site, and acquires a train of amplitudes indicated by the pulse wave signal;
a first envelope creation unit that creates, with respect to the train of amplitudes acquired by the pulse wave amplitude train acquisition unit, a first envelope connecting the amplitudes;
a local-extremum detection unit that detects local maxima and local minima in the first envelope;
a local-maximum envelope creation unit that creates, with respect to a train of amplitudes corresponding to the local maxima in the train of amplitudes acquired by the pulse wave amplitude train acquisition unit, a local-maximum envelope connecting the amplitudes on a plane where a cuff pressure and a pulse wave amplitude form orthogonal coordinates; and
a local-minimum envelope creation unit that creates, with respect to a train of amplitudes corresponding to the local minima in the train of amplitudes acquired by the pulse wave amplitude train acquisition unit, a local-minimum envelope connecting the amplitudes on the plane,
wherein the variation amount calculation unit obtains, as the blood pressure variation amount, a second difference or ratio between a cuff pressure at which the local-maximum envelope has a certain pulse wave amplitude and a cuff pressure at which the local-minimum envelope has the certain pulse wave amplitude on a high-pressure side with respect to maximum peaks of the local-maximum envelope and the local-minimum envelope or on a low-pressure side with respect to the maximum peaks of the local-maximum envelope and the local-minimum envelope on the plane.

5. The electronic blood pressure meter according to claim 1, further comprising:
a display; and
a display processing unit that performs processing of displaying the heart failure index on the display together with a calculation result of blood pressure according to the oscillometric method.

6. A heart failure detector for outputting an index relatively representing severity of heart failure, the heart failure detector comprising:
a cuff pressure control unit capable of changing a pressure of a cuff worn on a measurement site;
a pressure detection unit that detects a cuff pressure signal representing the pressure of the cuff;
a variation amount calculation unit that obtains a blood pressure variation amount synchronized with respiration based on the cuff pressure signal;
a correspondence relation storage unit that stores a predetermined correspondence relation between a blood pressure variation amount synchronized with respiration and a heart failure index that numerically represents relative severity of heart failure and corresponds to a value of a biomarker indicating severity of heart failure; and
an index output unit that refers to the correspondence relation stored in the correspondence relation storage unit and outputs the heart failure index corresponding to the blood pressure variation amount obtained by the variation amount calculation unit.
